(19) United States
(12) Patent Application Publication
(10) Pub. No.: US 2019/0254988 A1
(43) Pub. Date: Aug. 22, 2019

(54) USE OF NON CRYSTALLINE TERPENE ALCOHOLS FOR THE INHIBITION OF CRYSTALLIZATION OF CANNABINOIDS

(71) Applicant: Botanical Process Solutions LLC, Santa Barbara, CA (US)

(72) Inventor: Matthew A. Archibald, Santa Barbara, CA (US)

(21) Appl. No.: 16/277,508

(22) Filed: Feb. 15, 2019

Related U.S. Application Data
(60) Provisional application No. 62/710,369, filed on Feb. 16, 2018.

(51) Int. Cl. A61K 31/05; A61K 47/10

(52) U.S. Cl. CPC A61K 31/05; A61P 25/18; A61K 47/10

(57) ABSTRACT

Non-crystalline compositions comprising cannabidiol in a concentration greater than 30% w/w and at least one terpene alcohol are disclosed, along with related methods of manufacture and use.

### CROSS-REFERENCE TO RELATED APPLICATIONS

This application claims the benefit of and priority to U.S. Provisional Patent Application No. 62/710,369, filed Feb. 16, 2018, which is hereby incorporated by reference in its entirety.

### BACKGROUND

Cannabinoid concentrates are of great interest for their psychoactive and medicinal properties. However, challenges arise during the formulation of cannabinoids because the concentrates tend to crystallize at relatively low concentrations, which limits the amount of active substance that can be ingested in a given dose, increases the amount of time necessary to reach a desired biological uptake, and affects the modes of administration available. Further, this crystallization can inhibit the release of other compounds blended with the cannabinoid(s). For example, some cannabinoid concentrates are enriched with small amounts of terpenes/terpenoids that provide additional health benefits and/or fragrance. Publications describing compositions containing cannabinoids and terpenes/terpenoids include, for example, U.S. Pat. Nos. 9,730,911 and 9,375,417 and US Patent Pub. Nos. 2014/0271940 and 2013/0059018.

### SUMMARY

The present invention generally relates to the use of terpene alcohols to disrupt hydrogen bonding within high-concentration cannabinoid compositions, thereby inhibiting the crystallization of cannabinoids. In practice, terpene alcohols, such as those that are non-crystalline at temperatures between 5 degrees Celsius and 25 degrees Celsius, can be blended with cannabinoids by high shear mixing or sonication to produce substances that range from liquid to semi-solid having a concentration of cannabidiol greater than 30% w/w. Such compositions can be used to increase bioavailability of cannabinoids and decrease the number of doses required to achieve a therapeutic or desired effect. Alone or in combination with other compounds, CBD compositions may be used to treat addiction, depression, PTSD, anxiety, psychosis and numerous other ailments.

Cannabinoids that have at least two alcohol groups or at least one alcohol and one carboxylic acid group can form crystals because the alcohol groups and carboxylic acid groups allow for hydrogen bonding between cannabinoid molecules. However, when a terpene alcohol is added to a solution containing cannabinoids, electrostatic attraction between the alcohol group of the terpene alcohol and the —OH and/or carboxylic acid group of a cannabinoid molecule disrupts the hydrogen bonding that leads to crystal formation. This disruption of crystal formation is beneficial because bioavailability of compounds is generally increased by the administration of non-crystalline forms.

The compositions disclosed herein can be made free from glycerin and synthetic glycols, which tend to degrade into toxic compounds when heated. Additionally, it is possible to achieve all-natural compositions by using terpene alcohols derived from plant material.

In an aspect, a composition comprises cannabidiol (CBD) in a concentration greater than 30% w/w and at least one terpene alcohol, wherein the composition is non-crystalline at temperatures greater than or equal to 0 degrees Celsius, or greater than or equal to 5 degrees Celsius, or room temperature.

In an aspect, a composition consists essentially of cannabidiol (CBD) in a concentration greater than 30% w/w and at least one terpene alcohol, wherein the composition is non-crystalline at temperatures greater than or equal to 0 degrees Celsius, or greater than or equal to 5 degrees Celsius, or room temperature.

In an embodiment, a composition comprising or consisting essentially of CBD and at least one terpene alcohol does not comprise a molecule having a glycerin moiety.

In an aspect, a composition consists of cannabidiol (CBD) in a concentration greater than 30% w/w and at least one terpene alcohol, wherein the composition is non-crystalline at temperatures greater than or equal to 0 degrees Celsius, or greater than or equal to 5 degrees Celsius, or room temperature.

In an aspect, CBD is present in a composition disclosed herein in a concentration between 30% and 80% w/w, or between 35% and 75% w/w, or between 40% and 70% w/w, or between 40% and 60% w/w, or between 45% and 80%, or between 70% and 80% w/w. In an embodiment, CBD is present in a composition disclosed herein in a concentration greater than 35% w/w, or greater than 40% w/w, or greater than 45% w/w, or greater than 50% w/w, or greater than 60% w/w, or greater than 70% w/w, or greater than 80% w/w.

In an embodiment, at least one terpene alcohol is present in a composition disclosed herein in a concentration between 5% and 60% w/w, or between 10% and 50% w/w, or between 10% and 40% w/w, or between 10% and 30% w/w, or between 10% and 20% w/w, or between 20% and 60% w/w, or between 25% and 60% w/w, or between 30% and 60% w/w, or between 25% and 50% w/w, or between 30% and 50% w/w, or between 25% and 40% w/w. In an embodiment, at least one terpene alcohol is present in a concentration greater than 20% w/w, or greater than 25% w/w, or greater than 30% w/w. Exemplary terpene alcohols for use in the compositions disclosed herein include but are not limited to terpene alcohols selected from the group consisting of alpha-terpineol, beta-terpineol, gamma-terpineol, terpinen-4-ol, alpha-bisabolol, linalool, fenchol, guaiol, borneol, isoborneol, eucalyptol, phytol, geraniol, nerolidol, isopulegol, beta-eudesmol, pinanol, eudesm-7(11)-en-4-ol, nerol and combinations thereof. In an embodiment, the at least one terpene alcohol is a single terpene alcohol. In an embodiment, the at least one terpene alcohol is a mixture of two, three, four or more terpene alcohols.

In an embodiment, a composition disclosed herein further comprises at least one terpene. For example, the terpene may be selected from the group consisting of a monoterpene, a sesquiterpene, and combinations thereof. In an embodiment, the concentration of the terpene(s) is between 1% and 20% w/w, or between 2% and 15% w/w, or between 2.5% and 12% w/w, or between 3% and 10% w/w, or between 5% and 10% w/w

In an embodiment, a composition disclosed herein further comprises at least one additional cannabinoid, i.e., at least one cannabinoid in addition to cannabidiol (CBD). For example, the additional cannabinoid may be selected from the group consisting of Δ⁹-tetrahydrocannabinol (THC), cannabigerol (CBG), cannabichromene (CBC), cannabigerivarin (CBGV), tetrahydrocannabivarin (THCV), cannabidivarin (CBDV), cannabichromevarin (CBCV), cannabinoid derivatives, and combinations thereof. In an embodiment, the concentration of the additional cannabinoid(s) is between 30% and 80% w/w, or between 35% and 75% w/w, or between 40% and 70% w/w, or between 45% and 65% w/w, or between 50% and 60% w/w. In an embodiment the additional cannabinoid is THC in a concentration less than 9% w/w, or less than 6% w/w, or less than 3% w/w, or between 0.5% and 9% w/w, or between 3% and 9% w/w, or between 3% and 6% w/w.

In an embodiment, a composition disclosed herein further comprises at least one non-terpene alcohol. For example, the non-terpene alcohol may be selected from the group consisting of ethanol, glycol, diols, polyols, glycosides and combinations thereof.

In an aspect, a composition comprises cannabidiol (CBD) in a concentration greater than 30% w/w, at least one terpene alcohol, and nicotine. In an embodiment, the composition is non-crystalline at room temperature. In an embodiment, the nicotine is present as an acid, salt or freebase. In an embodiment, the nicotine is present in a concentration between 0.5% w/w and 10% w/w, or between 1% w/w and 8% w/w, or between 2% w/w and 5% w/w.

In an aspect, a composition comprises cannabidiol (CBD) in a concentration greater than 30% w/w, at least one terpene alcohol, and a compound selected from the group consisting of a dopamine re-uptake inhibitor, a dopamine releasing agent, an opioid-receptor agonist, an anti-depressant, an NMDA antagonist, a 5-hydroxy-tryptamine-receptor (5-HT₂-receptor or 5-HT_{2A}-receptor) agonist and combinations thereof.

In an aspect, a composition comprises cannabidiol (CBD) in a concentration greater than 30% w/w, at least one terpene alcohol, and a compound or moiety selected from the group consisting of methylphenidate, methamphetamine, amphetamine, mitragynine, 7-hydroxymitragynine, psilocin, salvinorin A, herkinorin, ketamine, esketamine and combinations thereof. In an embodiment, the compound or moiety is present as an acid, salt or freebase. In an embodiment, the compound is present in a concentration between 1×10⁻⁵% w/w and 10% w/w, or between 1×10⁻⁴% w/w and 5% w/w, or between 1×10⁻³% w/w and 1% w/w, or between 1×10⁻²% w/w and 0.1% w/w.

In an embodiment, a composition disclosed herein is a viscous liquid, a waxy solid or a semi-solid at room temperature.

In an embodiment, a composition disclosed herein is formulated as a tincture, a pill, a capsule, a suppository or a microencapsulated flowable powder. Such dosage forms may be prepared in accordance with standard principles of pharmaceutical formulation, known to those skilled in the art.

In an embodiment, a composition disclosed herein may include one or more physiologically acceptable carriers or excipients, such as fillers, diluents, disintegrants, surfactants, binders, glidants, and lubricants.

Suitable carriers include but are not limited to maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof).

Fillers suitable for use in the compositions disclosed herein include, but are not limited to: celluloses, modified celluloses, (e.g. sodium carboxymethyl cellulose, ethyl cellulose, hydroxymethyl cellulose, hydroxypropylcellulose), cellulose acetate, microcrystalline cellulose, calcium phosphates, dibasic calcium phosphate, starches (e.g. corn starch, potato starch), sugars (e.g., sorbitol, lactose, sucrose, or the like), or any combination thereof.

Diluents suitable for use in the compositions disclosed herein include, but are not limited to: sugars, for example, confectioner's sugar, compressible sugar, dextrates, dextrin, dextrose, lactose, mannitol, sorbitol, cellulose, and modified celluloses, for example, powdered cellulose, talc, calcium phosphate, starch, or any combination thereof.

Disintegrants suitable for use in the compositions disclosed herein include but are not limited to croscarmellose sodium, sodium starch glycolate, or a combination thereof.

Surfactants suitable for use in the compositions disclosed herein include but are not limited to sodium lauryl sulfate (SLS), sodium stearyl fumarate (SSF), polyoxyethylene 20 sorbitan mono-oleate (e.g., Tween™), or any combination thereof.

Binders suitable for use in the compositions disclosed herein include but are not limited to polyvinylpyrrolidone, dibasic calcium phosphate, sucrose, corn (maize) starch, modified cellulose (e.g., hydroxymethyl cellulose), or any combination thereof.

Glidants suitable for use in the compositions disclosed herein include but are not limited to colloidal silicon dioxide, talc, or a combination thereof.

Exemplary lubricants for use in the compositions disclosed herein include magnesium stearate, calcium stearate, zinc stearate, sodium stearate, stearic acid, aluminum stearate, leucine, hydrogenated vegetable oil or any combination thereof.

In an aspect, a method of using any one of the compositions disclosed herein comprises administering the composition to a subject in the form of a vapor, a pill, a capsule, a suppository or a microencapsulated flowable powder.

In an aspect, a method for making any one of the compositions disclosed herein comprises subjecting the composition to high shear mixing or sonication.

In an embodiment where the composition is subjected to high shear mixing, the composition is heated to a temperature between 150° F. and 220° F., or between 160° F. and 200° F., or between 175° F. and 185° F. In an embodiment where the composition is subjected to high shear mixing, the composition is mixed at a speed between 2,000 and 15,000 rpm, or between 3,000 and 14,000 rpm, or between 4,000 and 12,000 rpm, or between 5,000 and 10,000 rpm, or between 7,500 and 10,000 rpm. In an embodiment where the composition is subjected to high shear mixing, the composition is mixed for between 20 minutes and 3 hours, or between 30 minutes and 2 hours, or between 45 minutes and 90 minutes, or between 45 minutes and 60 minutes. In an embodiment where the composition is subjected to high shear mixing, the composition is heated to 180° F. and mixed at 10,000 rpm for up to 1 hour.

In an embodiment where the composition is sonicated, sonication occurs at a frequency between 20 KHz and 2.4 MHz, or between 20 KHz and 1 MHz, or between 20 KHz and 500 KHz, or between 20 KHz and 100 KHz, or between 20 KHz and 40 KHz. In an embodiment where the composition is sonicated, sonication occurs for between 1 hour and 10 hours, or between 2 hours and 10 hours, or between 4 hours and 10 hours, or between 6 hours and 10 hours, or between 8 hours and 10 hours. In an embodiment where the composition is sonicated, sonication occurs at a frequency between 20-40 KHz for up to 10 hours.

In an aspect, a method for increasing the adsorption of cannabidiol (CBD) into cells comprises administering a composition disclosed herein to a subject. For example, the subject may be a human, a feline, an equine, a bovine, a canine, a porcine, an ovine, a murine, a reptile, or an avian. In an embodiment, the composition is in the form of a vapor, a pill, a capsule, a suppository or a microencapsulated flowable powder. In an embodiment, the administration occurs by a route that bypasses the subject's liver, such as by vapor or suppository.

### BRIEF DESCRIPTION OF THE DRAWINGS

FIG. 1 graphically depicts the percent concentration of CBD versus crystallization scores for alpha bisabolol at 10% w/w, 20% w/w and 30% w/w and a control.

FIG. 2 graphically depicts the percent concentration of CBD versus crystallization scores for geraniol at 10% w/w, 20% w/w and 30% w/w and a control.

FIG. 3 graphically depicts the percent concentration of CBD versus crystallization scores for linalool at 10% w/w, 20% w/w and 30% w/w and a control.

FIG. 4 graphically depicts the percent concentration of CBD versus the calculated effectiveness of alpha bisabolol to reduce or prevent crystallization at various terpene alcohol concentrations.

FIG. 5 graphically depicts the percent concentration of CBD versus the calculated effectiveness of geraniol to reduce or prevent crystallization at various terpene alcohol concentrations.

FIG. 6 graphically depicts the percent concentration of CBD versus the calculated effectiveness of linalool to reduce or prevent crystallization at various terpene alcohol concentrations.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of this description.

As used herein, the term “physiologically acceptable carrier” refers to any carrier or excipient commonly used in compositions for human or animal consumption.

As used herein, an “excipient” refers to an inactive substance that serves as the vehicle or medium for an active substance.

As used herein, a “disintegrant” is an excipient that hydrates a composition and aids in tablet dispersion.

As used herein, a “diluent” or “filler” is an excipient that adds bulkiness to a composition.

As used herein, a “surfactant” is an excipient that imparts compositions with enhanced solubility and/or wetability.

As used herein, a “binder” is an excipient that imparts a composition with enhanced cohesion or tensile strength (e.g., hardness).

As used herein, a “glidant” is an excipient that imparts a composition with enhanced flow properties.

As used herein, a “colorant” is an excipient that imparts a composition with a desired color. Examples of colorants include commercially available pigments such as titanium dioxide, iron oxide, and/or combinations thereof.

As used herein, a “lubricant” is an excipient that is added to compositions that are pressed into tablets. The lubricant aids in compaction of granules into tablets and ejection of a tablet of a composition from a die press.

As used herein, the term “cannabinoid” refers to a chemical compound that shows direct or indirect activity at a cannabinoid receptor. There are two main cannabinoid receptors, CB₁ and CB₂. Other receptors that research suggests have cannabinoid activity include the GPR55 and GPR18 receptors. The term “phytocannabinoid” refers to cannabinoids that occur in a plant species or are derived from cannabinoids occurring in a plant species. Examples of cannabinoids include, but are not limited to, Tetrahydrocannabinol (THC), Cannabidiol (CBD), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), and Cannabigerol Monomethyl Ether (CBGM).

As used herein, the term “cannabinoid derivative” refers to a chemical compound having a structure similar to a cannabinoid but which includes at least one functional group that does not occur in nature.

As used herein, the term “terpene” refers to hydrocarbons of biological origin having carbon skeletons formally derived from isoprene [CH₂═C(CH₃)CH═CH₂]. This class is subdivided into the C5 hemiterpenes, C10 monoterpenes, C15 sesquiterpenes, C20 diterpenes, C25 sesterterpenes, C30 triterpenes, C40 tetraterpenes (carotenoids), and C5n polyterpenes.

As used herein, a “terpene alcohol” is a terpene molecule comprising at least one alcohol (—OH) group. In some embodiments, terpene alcohols that are non-crystalline around room temperature (i.e., 65-75 degrees Fahrenheit, herein referred to as “non-crystalline terpene alcohols”) are used in the disclosed compositions.

As used herein, “glycerin” refers to CH(OH)C(OH)CH(OH) and a “glycerin moiety” refers to a portion of a molecule that has the same general formula as glycerin with one or more of the alcoholic hydrogens removed. Glycerin itself comprises a glycerin moiety.

As used herein, the term “synthetic” refers to a molecule, which may or may not occur in nature, when the molecule is synthesized ex vivo by man from one or more starting materials. Compounds in the compositions disclosed herein may be derived from plant sources (e.g., phytocannabinoids) or synthetic.

As used herein, the term “high shear mixing” refers to a method of mixing components that moves adjacent areas of fluid at different relative velocities, for example, by use of a rotating impeller or high speed rotor and a stator.

As used herein, “non-crystalline” describes a state of matter lacking long-range order. For example, a “non-crystalline solid” may be amorphous or semi-crystalline.

As used herein, a “semi-solid” is a state of matter having physical properties similar to those of a solid (e.g., a self-supporting shape) but which contains a non-negligible amount of entrained liquid.

The terms “direct and indirect” describe the actions or physical positions of one component relative to another component, or one device relative to another device. For example, a component that “directly” acts upon or touches another component does so without intervention from an intermediary. Contrarily, a component that “indirectly” acts upon or touches another component does so through an intermediary (e.g., a third component).

Ratios of compounds are disclosed herein on a weight-by-weight basis (w/w). However, those of ordinary skill in the art are familiar with methods for converting w/w ratios into other units (e.g., moles, grams, weight-by-volume (w/v), etc.). When the density of liquid components in a composition is close to 1.0, w/w and w/v can be used interchangeably.

The use of compositions and methods disclosed herein is further illustrated by the following Examples. These Examples are for illustrative purposes only and are not intended to limit the invention.

Crystallization testing was performed on samples comprising various concentrations of CBD and terpene alcohols. Five different CBD concentrations dissolved in three different terpene alcohols at three terpene alcohol concentrations were evaluated and compared to a control group of the same CBD concentrations dissolved in nominally pure limonene. The results of the study support the use of terpene alcohols to disrupt the crystallization of high-concentration cannabinoid compositions.

Materials

Three terpene alcohols were selected: Linalool by John D. Walsh, cas#78-70-6% Purity 90%-100%, Geraniol by True Terpenes CAS#106-24-1 purity 70%, and Alpha Bisabolol by KIC Chemical Inc., CAS#23089-26-21, purity 97%. CBD was supplied by Nectartek LLC and had a COA purity of 99.7%. D-limonene was supplied by John D. Walsh, CAS#5989-27-5 and a purity of 100%. The sonicator was a Gemoro model 30 TH 1735 and has a frequency output of 40 KHz. The refrigerator was a Frigidaire Wine Refrigerator model #FFC38B2RS and the temperature tracking device used was the HOBO brand, model MX1101.

Method

After all materials were combined in the prescribed ratios, each sample was heated to 190° F. in a double boiler for 10 minutes and shaken vigorously by hand for 30 seconds.

All samples were then placed in an ultrasonic bath at 175° F. and 40 KHz for three hours. The vials were then placed in a temperature-controlled refrigerator at 44° F. with the temperature verified using a temperature tracker. The samples were visually inspected for crystals at day 7, 14 and 27.

At day 14, seed crystals of pure CBD were introduced into every sample and control vial, and on day 27 the vials were visually inspected for crystal formation.

Three replications of 5 different concentrations of CBD (30%, 40%, 50%, 60%, and 70%) mixed with 3 concentrations (10%, 20%, 30%) of the three terpene alcohols (alpha bisabolol, geraniol, linalool) with limonene as the inactive diluent were tested. The criteria for non-crystallization was established at zero visual crystallization evident. A scoring system for crystallization was used from slight: 1 to heavy: 5. The score was assigned at the end of each week. To tabulate the score, the individual scores were added together within each CBD concentration replication to represent the degree of crystallization.

### Example 1

Alpha bisabolol sample mixing schedules and a control group schedule are shown in Tables 1-3 and 4, respectively.

TABLE 1
AB1: 10% Alpha Bisabolol
3 Samples each for every % concentration CBD

| | CBD Concentration: | | | | |
|---|---|---|---|---|---|
| Ingredient | 30% | 40% | 50% | 60% | 70% |
| CBD | 1.5 | 2.0 | 2.5 | 3.0 | 3.5 |
| Limonene | 3.00 | 2.50 | 2.00 | 1.50 | 1.00 |
| Alpha Bisabolol | 0.5 | 0.5 | 0.5 | 0.5 | 0.5 |
| Total | 5.0 | 5.0 | 5.0 | 5.0 | 5.0 |

TABLE 2
AB2: 20% Alpha Bisabolol
3 Samples each for every % concentration CBD

| | CBD Concentration: | | | | |
|---|---|---|---|---|---|
| Ingredient | 30% | 40% | 50% | 60% | 70% |
| CBD | 1.5 | 2.0 | 2.5 | 3.0 | 3.5 |
| Limonene | 2.50 | 2.00 | 1.50 | 1.00 | 0.50 |
| Alpha Bisabolol | 1 | 1 | 1 | 1 | 1 |
| Total | 5.0 | 5.0 | 5.0 | 5.0 | 5.0 |

TABLE 3
AB3: 30% Alpha Bisabolol
3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 2.0 | 1.5 | 1.0 | 0.5 | 0.0 |
| Alpha Bisabolol | 1.5 | 1.5 | 1.5 | 1.5 | 1.5 |
| Total | 5.0 | 5.0 | 5.0 | 5.0 | 5.0 |

TABLE 4
CG1: Single Sample Each % Concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 3.5 | 3.0 | 2.5 | 2.0 | 1.5 |
| Total | 5.0 | 5.0 | 5.0 | 5.0 | 5.0 |

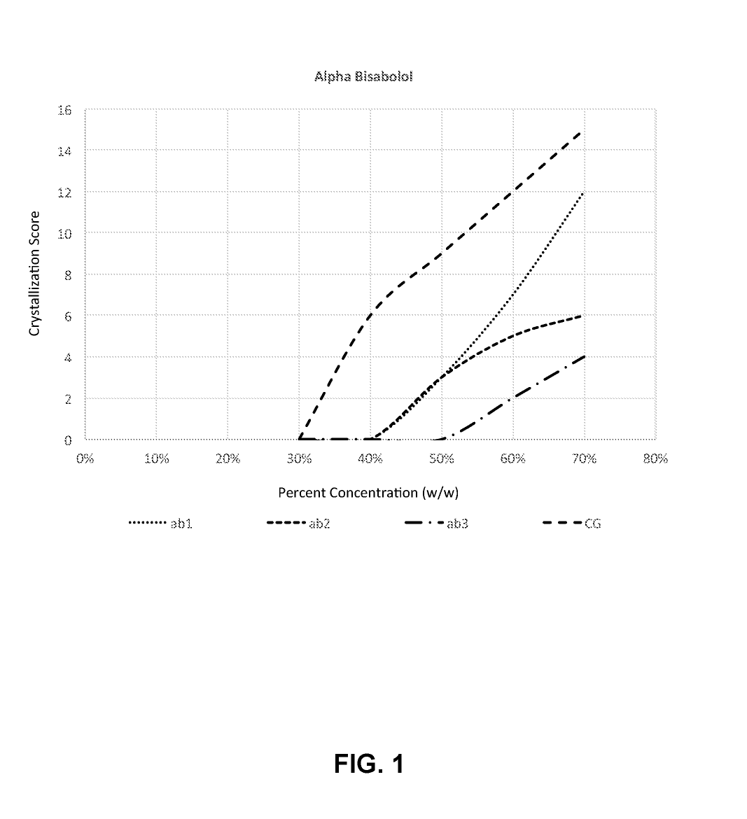

Crystallization scores for the alpha bisabolol samples are shown in Tables 5-7, and FIG. 1 graphically depicts the percent concentration of CBD versus crystallization scores for alpha bisabolol at 10% w/w, 20% w/w and 30% w/w and a control.

TABLE 5
AB1: 10% Alpha Bisabolol

| % CBD | 30% | | | 40% | | | 50% | | | 60% | | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 1 | 1 | 0 | 2 | 0 |
| Day 14 | 0 | 0 | 0 | 0 | 0 | 0 | 0 | 0 | 0 | 0 | 1 | 1 | 1 | 2 | 0 |
| Day 27 | 0 | 0 | 0 | 0 | 0 | 0 | 1 | 1 | 1 | 2 | 2 | 2 | 2 | 2 | 2 |

| | | | | | |
|---|---|---|---|---|---|
| Total Score Day 27 | 0 | 0 | 3 | 6 | 6 |

TABLE 6
AB2 = 20% Alpha Bisabolol

| % CBD | 30% | | | 40% | | | 50% | | | | 60% | | 70% | | |
|---|---|---|---|---|---|---|---|---|---|---|---|---|---|---|---|
| Day 07 | 0 | 0 | 0 | 0 | 0 | 0 | 0 | 0 | 0 | 1 | 0 | 1 | 1 | 0 | 1 |
| Day 14 | 0 | 0 | 0 | 0 | 0 | 0 | 0 | 0 | 0 | 1 | 1 | 0 | 2 | 0 | 2 |
| Day 27 | 0 | 0 | 0 | 0 | 0 | 0 | 1 | 1 | 1 | 1 | 2 | 2 | 2 | 2 | 2 |

| | | | | | |
|---|---|---|---|---|---|
| Total Score Day 27 | 0 | 0 | 3 | 5 | 6 |

TABLE 7
AB3 = 30% Alpha Bisabolol

| % CBD | 30% | | | 40% | | | 50% | | | | 60% | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 0 | 0 | 0 | 1 | 0 | 1 | 1 | 2 | 2 |

| | | | | | |
|---|---|---|---|---|---|
| Total Score Day 27 | 0 | 0 | 0 | 2 | 5 |

### Example 2

Geraniol sample mixing schedules and a control group schedule are shown in Tables 8-10 and 11, respectively.

TABLE 8
G1: 10% - 3 Samples each for every % concentration CBD

| | CBD Percentage | | | | |
|---|---|---|---|---|---|
| Ingredient | 30% | 40% | 50% | 60% | 70% |
| CBD | 1.5 | 2.0 | 2.5 | 3.0 | 3.5 |
| Limonene | 3.00 | 2.50 | 2.00 | 1.50 | 1.00 |
| Geraniol | 0.5 | 0.5 | 0.5 | 0.5 | 0.5 |
| Total | 5.0 | 5.0 | 5.0 | 5.0 | 5.0 |

TABLE 9
G2: 20% - 3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 2.50 | 2.00 | 1.50 | 1.00 | 0.50 |
| Geraniol | 1 | 1 | 1 | 1 | 1 |
| Total | 5.0 | 5.0 | 5.0 | 5.0 | 5.0 |

TABLE 10
G3: 30% - 3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 2.00 | 1.50 | 1.00 | 0.50 | 0.00 |
| Geraniol | 1.5 | 1.5 | 1.5 | 1.5 | 1.5 |
| Total | 5.0 | 5.0 | 5.0 | 5.0 | 5.0 |

TABLE 11
CG2: Single Sample Each % Concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 3.5 | 3.0 | 2.5 | 2.0 | 1.5 |
| Total | 5.0 | 5.0 | 5.0 | 5.0 | 5.0 |

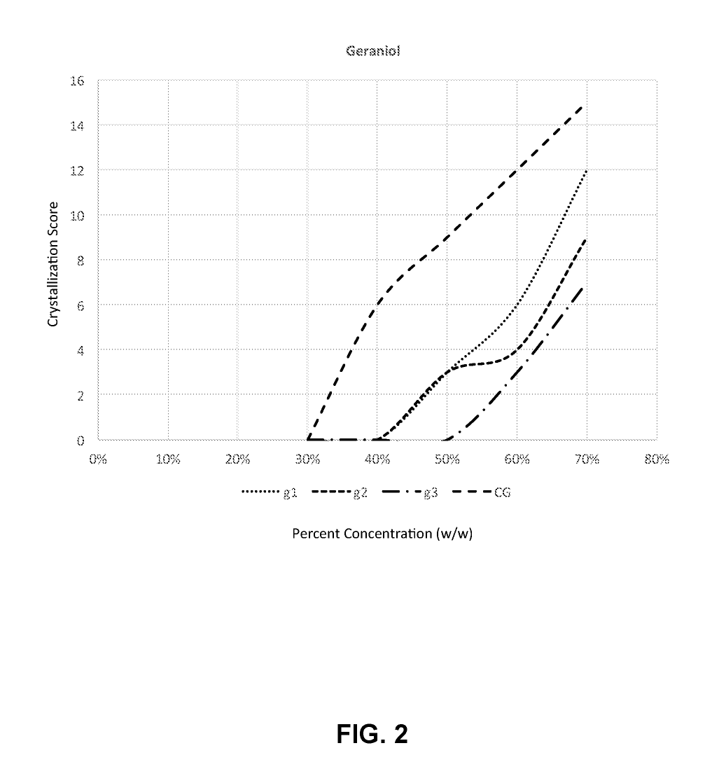

Crystallization scores for the geraniol samples are shown in Tables 12-14, and FIG. 2 graphically depicts the percent concentration of CBD versus crystallization scores for geraniol at 10% w/w, 20% w/w and 30% w/w and a control.

TABLE 12
G1 = 10% Geraniol

| % CBD | 30% | | | 40% | | | 50% | | | 60% | | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 1 | 1 | 1 | 2 | 2 | 2 | 4 | 4 | 4 |

| | | | | | |
|---|---|---|---|---|---|
| Total Score Day 27 | 0 | 0 | 3 | 6 | 12 |

TABLE 13
G2 = 20% Geraniol

| % CBD | 30% | | | 40% | | | 50% | | | | 60% | | 70% | | |
|---|---|---|---|---|---|---|---|---|---|---|---|---|---|---|---|
| Day 07 | 0 | 0 | 0 | 0 | 0 | 0 | 0 | 0 | 0 | 1 | 1 | 1 | 0 | 2 | 2 |
| Day 14 | 0 | 0 | 0 | 0 | 0 | 0 | 0 | 0 | 0 | 1 | 1 | 1 | 2 | 0 | 2 |
| Day 27 | 0 | 0 | 0 | 0 | 0 | 0 | 1 | 1 | 1 | 1 | 1 | 2 | 3 | 3 | 3 |

| | | | | | |
|---|---|---|---|---|---|
| Total Score Day 27 | 0 | 0 | 3 | 4 | 9 |

TABLE 14
G3 = 30% Geraniol

| % CBD | 30% | | | 40% | | | 50% | | | 60% | | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 0 | 0 | 0 | 1 | 1 | 1 | 2 | 3 | 2 |

| | | | | | |
|---|---|---|---|---|---|
| Total Score Day 27 | 0 | 0 | 0 | 3 | 9 |

### Example 3

Linalool sample mixing schedules and a control group schedule are shown in Tables 15-17 and 18, respectively.

TABLE 15
L1: 10% - 3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 3.00 | 2.50 | 2.00 | 1.50 | 1.00 |
| Linalool | 0.5 | 0.5 | 0.5 | 0.5 | 0.5 |
| Total | 5.0 | 5.0 | 5.0 | 5.0 | 5.0 |

TABLE 16
L2: 20% - 3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 2.50 | 2.00 | 1.50 | 1.00 | 0.50 |
| Linalool | 1 | 1 | 1 | 1 | 1 |
| Total | 5.0 | 5.0 | 5.0 | 5.0 | 5.0 |

TABLE 17
L3: 30% - 3 Samples each for every % concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 2.00 | 1.50 | 1.00 | 0.50 | 0.00 |
| Linalool | 1.5 | 1.5 | 1.5 | 1.5 | 1.5 |
| Total | 5.0 | 5.0 | 5.0 | 5.0 | 5.0 |

TABLE 18
CG3: Single Sample Each % Concentration CBD

| Ingredient | 30% | 40% | 50% | 60% | 70% |
|---|---|---|---|---|---|
| CBD | 1.5 | 2.0 | 2.5 | 3.0 | 3.5 |
| Limonene | 3.5 | 3.0 | 2.5 | 2.0 | 1.5 |
| Total | 5.0 | 5.0 | 5.0 | 5.0 | 5.0 |

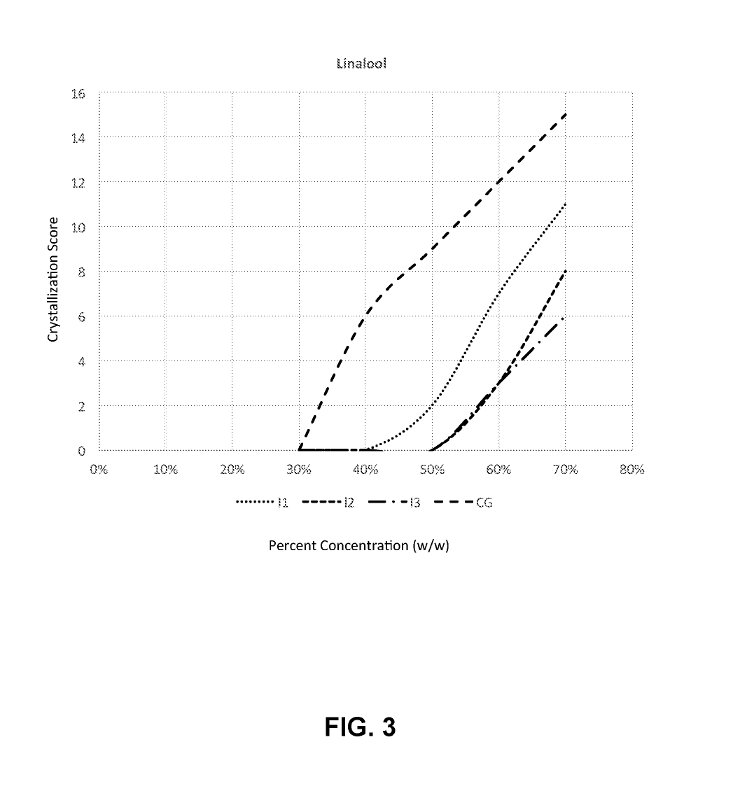

Crystallization scores for the linalool samples are shown in Tables 19-21, and FIG. 3 graphically depicts the percent concentration of CBD versus crystallization scores for linalool at 10% w/w, 20% w/w and 30% w/w and a control.

TABLE 19
L1 = Linalool 10%

| % CBD | 30% | | | 40% | | | 50% | | | 60% | | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 0 | 1 | 1 | 1 | 2 | 2 | 3 | 4 | 4 |

| | | | | | |
|---|---|---|---|---|---|
| Total Score Day 27 | 0 | 0 | 2 | 4 | 11 |

TABLE 20
L2 = Linalool 20%

| % CBD | 30% | | | 40% | | | 50% | | | 60% | | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 0 | 0 | 0 | 1 | 1 | 1 | 3 | 2 | 3 |

| | | | | | |
|---|---|---|---|---|---|
| Total Score Day 27 | 0 | 0 | 0 | 3 | 8 |

TABLE 21
L3 = Linalool 30%

| % CBD | 30% | | | 40% | | | 50% | | | | 60% | | 70% | | |
|---|---|---|---|---|---|---|---|---|---|---|---|---|---|---|---|
| Day 07 | 0 | 0 | 0 | 0 | 0 | 0 | 0 | 0 | 0 | 0 | 0 | 0 | 0 | 0 | 0 |
| Day 14 | 0 | 0 | 0 | 0 | 0 | 0 | 0 | 0 | 0 | 0 | 0 | 0 | 0 | 0 | 0 |
| Day 27 | 0 | 0 | 0 | 0 | 0 | 0 | 0 | 0 | 0 | 1 | 1 | 1 | 2 | 2 | 2 |

| | | | | | |
|---|---|---|---|---|---|
| Total Score Day 27 | 0 | 0 | 0 | 3 | 6 |

### Example 4

Crystallization scores for the control group are shown in Table 22.

TABLE 22

| | CBD % | | | | |
|---|---|---|---|---|---|
| | 30% | 40% | 50% | 60% | 70% |
| Day 07 | 0 | 0 | 0 | 0 | 0 |
| Day 14 | 0 | 0 | 0 | 0 | 0 |
| Day 27 | 0 | 6 | 9 | 12 | 15 |

Effectiveness scores representing each terpene alcohol's ability to prevent or reduce crystallization compared to the control group was determined by dividing the summed crystallization score at each terpene alcohol concentration by the Day 27 CG score in Table 22 and subtracting the resulting percentage from 100%. A sample score of zero was considered 100% effective for the prevention of crystallization, except when the control group score was also zero. Results are shown in Tables 23-25.

TABLE 23
Alpha Bisabolol

| | CBD % | | | | |
|---|---|---|---|---|---|
| | 30% | 40% | 50% | 60% | 70% |
| ab1 | 0 | 0 | 3 | 7 | 12 |
| Effectiveness: | n/a | 100% | 67% | 42% | 20% |
| ab2 | 0 | 0 | 3 | 5 | 6 |
| Effectiveness: | n/a | 100% | 67% | 58% | 60% |
| ab3 | 0 | 0 | 0 | 2 | 4 |
| Effectiveness: | n/a | 100% | 100% | 83% | 73% |
| CG | 0 | 6 | 9 | 12 | 15 |

TABLE 24
Geraniol

| | CBD % | | | | |
|---|---|---|---|---|---|
| | 30% | 40% | 50% | 60% | 70% |
| g1 | 0 | 0 | 0 | 6 | 12 |
| Effectiveness: | n/a | 100% | 100% | 50% | 20% |
| g2 | 0 | 0 | 3 | 3 | 9 |
| Effectiveness: | n/a | 100% | 67% | 75% | 40% |
| g3 | 0 | 0 | 0 | 3 | 7 |
| Effectiveness: | n/a | 100% | 100% | 75% | 53% |
| CG | 0 | 6 | 9 | 12 | 15 |

TABLE 25
Linalool

| | CBD % | | | | |
|---|---|---|---|---|---|
| | 30% | 40% | 50% | 60% | 70% |
| l1 | 0 | 0 | 2 | 7 | 11 |
| Effectiveness: | n/a | 100% | 78% | 42% | 27% |
| l2 | 0 | 0 | 0 | 3 | 8 |
| Effectiveness: | n/a | 100% | 100% | 75% | 47% |
| l3 | 0 | 0 | 0 | 3 | 6 |
| Effectiveness: | n/a | 100% | 100% | 75% | 60% |
| CG | 0 | 6 | 9 | 12 | 15 |

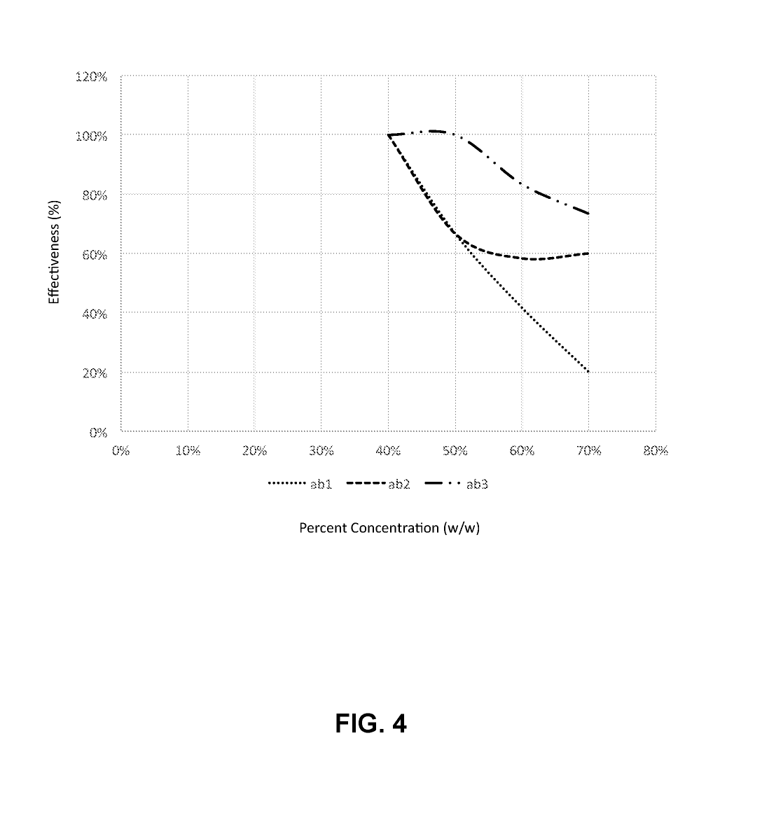

FIG. 4 graphically depicts the percent concentration of CBD versus the calculated effectiveness of alpha bisabolol to reduce or prevent crystallization at various terpene alcohol concentrations.

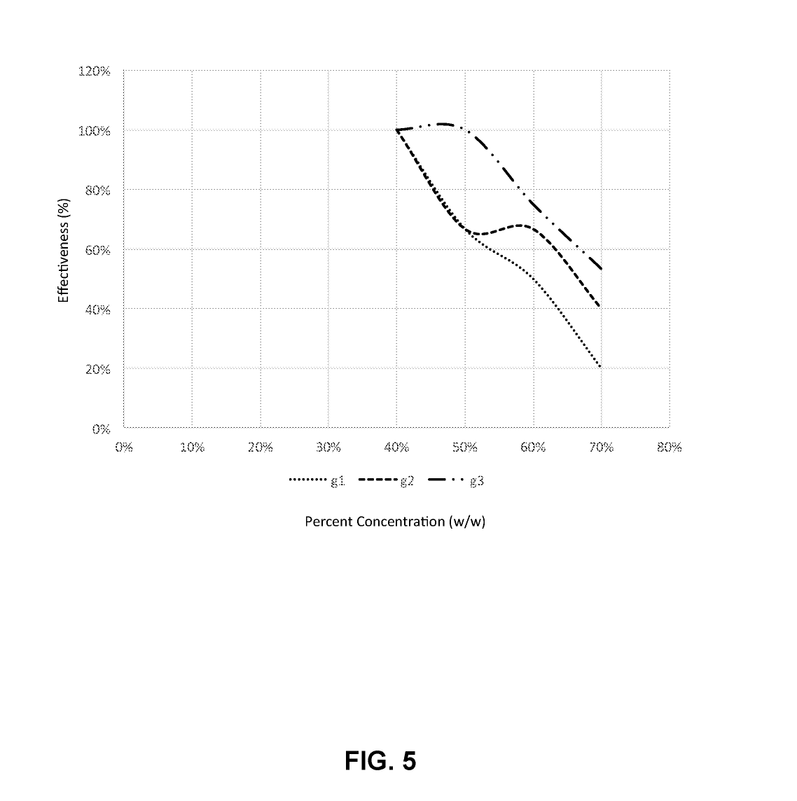

FIG. 5 graphically depicts the percent concentration of CBD versus the calculated effectiveness of geraniol to reduce or prevent crystallization at various terpene alcohol concentrations.

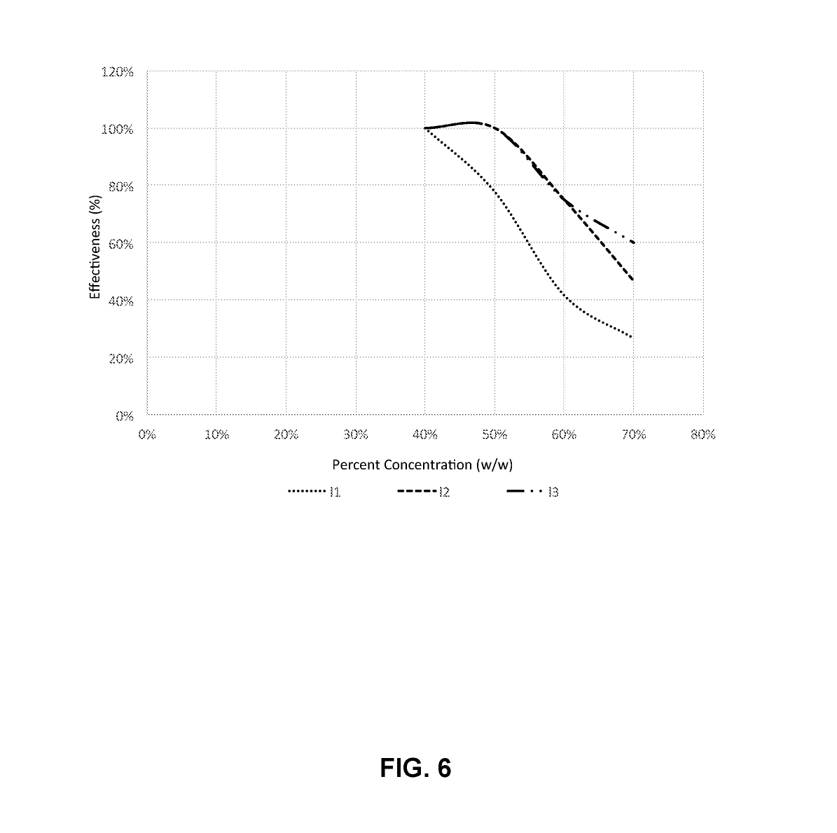

FIG. 6 graphically depicts the percent concentration of CBD versus the calculated effectiveness of linalool to reduce or prevent crystallization at various terpene alcohol concentrations.

Results and Discussion

The addition of terpene alcohols to CBD mixtures decreases or eliminates the incidence of crystallization compared to the control group when the terpene alcohol is present at a concentration between 10%-30% w/w and the CBD concentration is above 30% w/w. At 50% CBD and 30% terpene alcohol, there were no incidences of crystallization compared to moderately heavy crystallization in the control. Thus, terpene alcohols significantly reduce or eliminate crystallization, and the cool temperatures utilized in the testing protocol indicate product stability even at low temperatures.

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments, exemplary embodiments and optional features, modification and variation of the concepts herein disclosed can be resorted to by those skilled in the art, and that such modifications and variations are considered to be within the scope of this invention as defined by the appended claims. The specific embodiments provided herein are examples of useful embodiments of the invention and it will be apparent to one skilled in the art that the invention can be carried out using a large number of variations of the devices, device components, and method steps set forth in the present description. As will be apparent to one of skill in the art, methods and devices useful for the present methods and devices can include a large number of optional composition and processing elements and steps. All art-known functional equivalents of materials and methods are intended to be included in this disclosure.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure.

It must be noted that as used herein and in the appended claims, the singular forms “a”, “an”, and “the” include plural reference unless the context clearly dictates otherwise. Thus, for example, reference to “a molecule” includes a plurality of such molecules and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range. As used herein, ranges specifically include all the integer values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is/are not specifically disclosed herein.

What is claimed is:

1. A composition comprising:
cannabidiol (CBD) in a concentration greater than 30% w/w; and
at least one terpene alcohol;
wherein the composition is non-crystalline at temperatures greater than or equal to 0 degrees Celsius.

2. The composition of claim 1, wherein the composition does not comprise a molecule having a glycerin moiety.

3. The composition of claim 1, wherein the CBD is present in a concentration between 30% and 80% w/w.

4. The composition of claim 1, wherein the at least one terpene alcohol is present in a concentration between 5% and 60% w/w.

5. The composition of claim 1, wherein the at least one terpene alcohol is present in a concentration greater than 20% w/w.

6. The composition of claim 4, wherein the terpene alcohol is selected from the group consisting of alpha-terpineol, beta-terpineol, gamma-terpineol, terpinen-4-ol, alpha-bisabolol, linalool, fenchol, guaiol, borneol, isoborneol, eucalyptol, phytol, geraniol, nerolidol, isopulegol, beta-eudesmol, pinanol, eudesm-7(11)-en-4-ol, nerol and combinations thereof.

7. The composition of claim 1 further comprising at least one terpene.

8. The composition of claim 7, wherein the terpene is selected from the group consisting of a monoterpene, a sesquiterpene, and combinations thereof.

9. The composition of claim 7, wherein the concentration of the terpene is between 1% and 20% w/w.

10. The composition of claim 1 further comprising at least one additional cannabinoid.

11. The composition of claim 10, wherein the additional cannabinoid is selected from the group consisting of Δ9-tetrahydrocannabinol (THC), cannabigerol (CBG), cannabichromene (CBC), cannabigerivarin (CBGV), tetrahydrocannabivarin (THCV), cannabidivarin (CBDV), cannabichromevarin (CBCV), cannabinoid derivatives, and combinations thereof.

12. The composition of claim 1 further comprising nicotine.

13. The composition of claim 1 further comprising a compound selected from the group consisting of methylphenidate, methamphetamine, amphetamine, mitragynine, 7-hydroxymitragynine, psilocin, salvinorin A, herkinorin, ketamine, esketamine and combinations thereof.

14. The composition of claim 1, wherein the composition is a viscous liquid.

15. The composition of claim 1, wherein the composition is formulated as a tincture, a pill, a capsule, a suppository or a microencapsulated flowable powder.

16. A method of using the composition of claim 1, the method comprising administering the composition to a subject in the form of a vapor, a pill, a capsule, a suppository or a microencapsulated flowable powder.

17. The method of claim 16, wherein the administration occurs by a route that bypasses the subject's liver.

18. A method for making the composition of claim 1, the method comprising subjecting the composition to high shear mixing or sonication.

19. The method of claim 18, wherein the composition is subjected to high shear mixing and the composition is heated to a temperature between 150° F. and 220° F. and mixed at a speed between 2,000-15,000 rpm for between 20 minutes and 3 hours.

20. The method of claim 18, wherein the composition is sonicated at a frequency between 20 KHz and 2.4 MHz for between 1 hour and 10 hours.
